SEQUENCE LISTING (6 sequences)

SEQ ID NO: 1 - DNA, 91 bp, Artificial sequence
  Synthetic Oligonucleotide
aagaaaccatgtagtttgtattcgaatgtcctatcctcagctgtcagaactcacctgttagacgtcgatctctctctagtggaagttagct

SEQ ID NO: 2 - DNA, 44 bp, Artificial sequence
  Synthetic oligonucleotide
atacaaactacatggtttcttagctaacttccactagagagaga

SEQ ID NO: 3 - DNA, 90 bp, Artificial sequence
  Synthetic oligonucleotide
aagaaaccatgtagtttgtattcgaatgtcctatcctcagctgtcagaactcacctgttagacgtcgatctctctctagtggaagttagc

SEQ ID NO: 4 - DNA, 44 bp, Artificial sequence; identical to SEQ ID NO: 2
  Synthetic oligonucleotide

SEQ ID NO: 5 - DNA, 22 bp, Artificial sequence
  Synthetic oligonucleotide
cagctgaggataggacattcga

SEQ ID NO: 6 - DNA, 21 bp, Artificial Sequence
  Synthetic oligonucleotide
tcagaactcacctgttagacg

(19) United States
(12) Patent Application Publication
(10) Pub. No.: US 2004/0048292 A1
(43) Pub. Date: Mar. 11, 2004

(54) Dual fidelity erca detection systems

(76) Inventors: Clifford Smith, Buchinghamshire (GB); Imogen Horsey, Buckinghamshire (GB); Tim Knott, Buckinghamshire (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/466,579

(22) PCT Filed: Jan. 15, 2002
(86) PCT No.: PCT/GB02/00125

(30) Foreign Application Priority Data
Jan. 19, 2001 (GB) 0101402.6

(51) Int. Cl. C12Q 1/68; C07H 21/04

(52) U.S. Cl. 435/6; 536/24.3

(57) ABSTRACT

The invention discloses an oligonucleotide probe which can be used for the identification of polymorphisms in a nucleic acid sequence. The probe contains an allele specific base at the 3′ end of the oligonucleotide and an additional deliberate mismatch base at a position of up to 4 nucleotides from the 3′ end of the probe. The oligonucleotide is particularly useful in Rolling Circle Amplification (RCA) reactions by increasing the specificity of discrimination between alleles.

### FIELD OF INVENTION

This invention relates to the area of nucleic acid analysis and in particular the analysis of differences in nucleic acid sequences.

### BACKGROUND

The requirement for examining genomes or fragments of DNA for polymorphisms especially single nucleotide polymorphisms (SNP) has increased rapidly with the discovery of more and more disease related genes. Currently the cost of sequencing in both time taken and reagents used is prohibitive to its use in genome screening. Genome screening is a very complex task due to the large number of possible mutations and the diverse relationship of these mutations to disease. A fast and cost effective system for analysing differences in nucleic acid sequences is essential for the comprehensive genome screens required for diagnostic and research purposes.

A number of methods have been described that enable extremely sensitive diagnostic assays based on nucleic acid detection. The majority employ exponential amplification of target or probe sequences. They include the polymerase chain reaction (PCR), ligase chain reaction (LCR), self-sustained sequence replication (3SR), nucleic acid sequence based amplification (ASBA), strand displacement amplification (SDA), and rolling circle amplification (RCA) WO 97/19193.

RCA based amplification methods involve DNA ligation, signal amplification from circular DNA and detection steps. The DNA ligation operation circularizes a specially designed nucleic acid probe molecule. This step is dependent on hybridization of the probe to a target sequence and results in the formation of circular probe molecules in proportion to the amount of target sequence present in a sample. The amplification operation occurs via rolling circle replication of the circularized probe. This is mediated via a single primer and a DNA polymerase, which may be processive and strand-displacing, resulting in a large amplification of the circularized probe sequences. Optionally, an additional amplification operation can be performed on the single stranded DNA product of rolling circle replication. Hyper-branched or exponential RCA [ERCA] is an extension of the basic RCA reaction that employs additional oligonucleotide primers to replicate the primary amplification product. This directs exponential syntheesis of branched double stranded DNA products and unsurpassed levels of amplification—amplification in excess of 10⁹ fold, Lizardi et al., Nature Genetics, Volume 19, July 1998, pp225-232.

Following RCA, the amplified probe sequences can be detected and quantified using any of the conventional detection systems for nucleic acids such as detection of fluorescent labels, enzyme-linked detection systems, antibody-mediated label detection, and detection of radioactive labels and electrochemical detection.

The viability of a nucleic acid based assay hinges on its accuracy and robustness when presented with just a few molecules of target. All of the above amplification methods offer good sensitivity, with a practical limit of detection of about 10-100 target molecules. Irrespective of the method used, it is crucial that the process is highly specific since the amplification of untargeted sequences can potentially limit reliability. For example, each RCA reaction is capable of rapidly generating and amplifying non-specific or spurious background signals.

The specificity of the current RCA assay relies on the fidelity of the ligase and the accuracy of hybridisation. The error rate from the ligation of 3′ mismatches varies with the ligase used, the bases involved in the mismatch, and the sequence context surrounding the mismatch, Luo et al., Nucleic Acids Research, 1996, Volume 24, No. 14, pp3071-3078, Housby et al., Nucleic Acids Research, 1998, Volume 26, pp4259-426 and in some cases is significant This coupled with the sensitivity of the RCA amplification affects the specificity of the reaction and hence any associated assay.

The present invention provides means for improving the specificity of the RCA assay by improving mismatch discrimination

### SUMMARY OF THE INVENTION

The primary objective of this invention is to improve the sensitivity and specificity of RCA based nucleic acid amplification by reducing or eliminating non-specific background signals particularly those due to errors at the ligation stage of the process.

The invention utilises the mismatch discrimination properties of two different enzymes as a strong selection against mis-ligation events. The invention provides novel probes which can be used to improve the fidelity of such assays, the probes being designed to include a mismatch and leave a gap when hybridised. Use can also be made of nucleotide analogues to further improve the fidelity of the assay.

### DESCRIPTION OF DRAWINGS

FIG. 1 shows the principle of the hybridisation and ligation steps of the RCA assay, Banér et al., Nucleic Acids Research, 1998, Volume 26, No 22, pp5073-5078.

FIG. 2 shows the principle of the exponential RCA (ERCA) reaction.

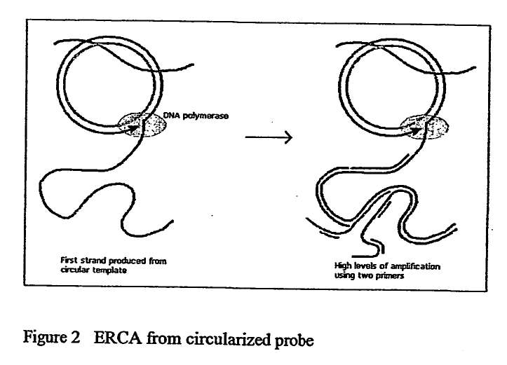

FIG. 3. Structure of 3′ amino dTTP.

FIG. 4. Assay Schematic. This example outlines the process for one of a pair of probes designed to score an A/G polymorphism. The ‘A’ probe reaction scheme is drawn.

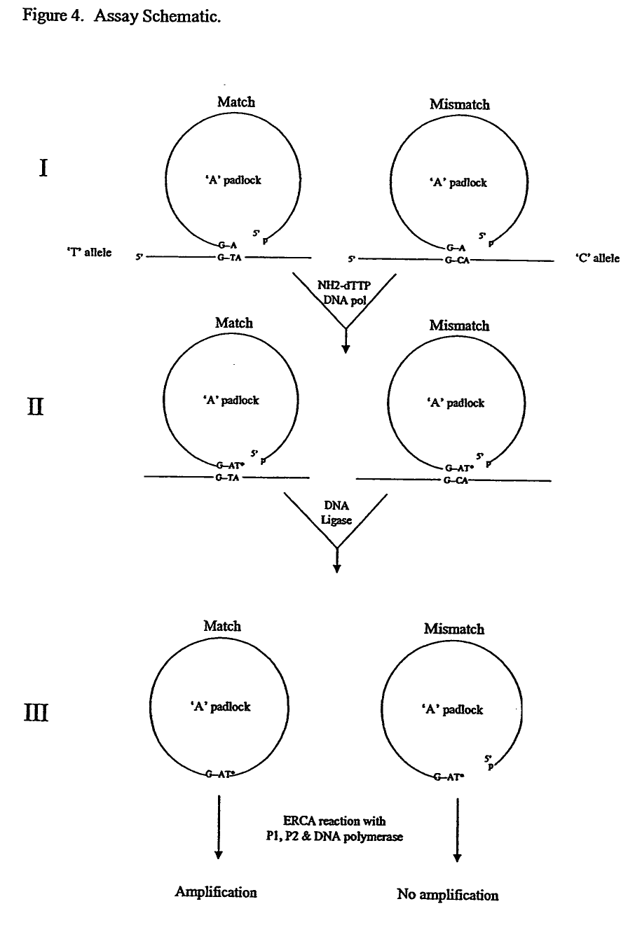

### DETAILED DESCRIPTION OF THE INVENTION

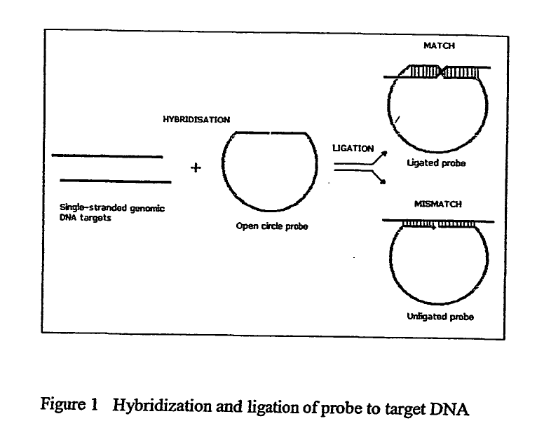

FIG. 1 shows the principle of the hybridisation and ligation steps of the RCA assay. A single stranded oligonucleotide probe hybridises with the target nucleic acid sequence. The target nucleic acid sequence can be made single stranded if necessary. Several bases at both the 3′ and 5′ ends of the probe hybridise to adjacent regions of the target nucleic acid sequence. The actual base at the 3′ end of the probe is specific to the SNP being investigated. If the correct base is present at the 3′ end, then the 3′ end and 5′ end of the hybridised probe will be readily ligated by a DNA ligase. A correct base is one that is complementary to the polymorphic base in the target nucleic acid sequence. If the correct base is not present at the 3′ end of the probe then ligation of the 3′ and 5′ ends of the hybridised probe will be much less efficient than if the 3′ base is complementary to the polymorphic base. Regions between the hybridised ligated 3′ and 5′ ends form an open circular structure that can be later amplified by RCA. However, even inefficient mismatch ligations can produce small quantities of circular amplifiable material so that even in these cases amplification can occur even if the incorrect base is present at the SNP site.

This invention addresses problems inherent in the current RCA and ERCA methods as outlined above in particular, problems at the ligation step. The invention involves the use of a deliberate mismatch base included within a few bases of the 3′ end of the oligonucleotide probe and the effect this has on two different enzymes (a DNA polymerase and DNA ligase) during subsequent reactions. This provides a strong selection against mis-ligation events. Use can also be made of nucleotide analogues to further improve the fidelity of the assay.

According to a first aspect, the invention provides novel probes. As with standard probes that have been used in RCA reactions the 3′ end base of the probe according to the invention is specific to the SNP of interest. This is an important feature as it allows probes according to the invention to be used in conjunction with generic ERCA primers targeted to the probe backbone. In one preferred embodiment, probes of the invention include a deliberate mismatched base located 2, 3 or 4 bases from the 3′ end. This increases the specificity of the subsequent primer extension reaction by DNA polymerase. The position and nature of this mismatch can be optimised for maximum discrimination using information available in the literature and known to one skilled in the art.

An alternative embodiment of the first aspect of the invention provides probes such that, when hybridised to the target sequence, there is a gap between the 5′ and 3′ termini of the probe (i.e.—the two locus specific arms of the probe). This gap is important in that it enables the 3′ and 5′ arms of the probe to hybridise independently without duplex stabilising stacking interactions. This gap is suitably 1 to 10 bases long. A gap of 2, 3 or 4 bases is particularly suitable and a single base gap is preferred. In a conventional, non-gapped probe the two arms butt up to each other and they behave as though they are covalently attached resulting in a very stable duplex—even if the 3′ base is mismatched This increases the likelihood of mis-ligation events in prior art probes. The probe 5′ end is synthesised with a 5′ phosphate.

In a particularly preferred embodiment the present invention provides a probe which includes a deliberate mismatch a few bases from its 3′ end and is designed such that when hybridised to the target sequence there is a gap between the 5′ and 3′ ends of at least 1 nucleotide.

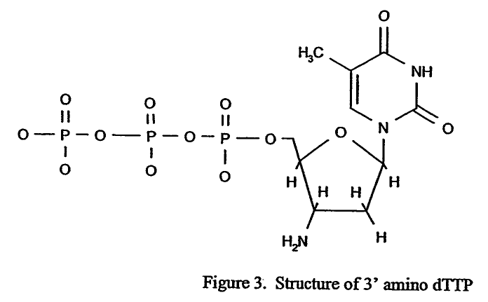

In a second aspect, the invention provides a method for determining the identity of a base at a specific site in a target nucleic acid by employing probes according to the invention in an RCA or ERCA assay. In embodiments where the probe has a gap, after hybridisation to the target sequence, the gap between the 3′ and 5′ ends of the probe is then filled by DNA polymerase activity. The identity of the base or bases in the gap will be known and so the synthesis can be done in a manner which prevents synthesis beyond the 5′ end of the probe or displacement of the 5′ end of the probe when the correct base is present at the SNP site. This can be achieved in-several ways and is illustrated in FIG. 3. In the preferred embodiment when the gap is a single nucleotide, the gap is filled with a nucleotide that acts as a terminator for further DNA polymerase activity when incorporated onto the 3′ end of the probe. An essential feature of this terminator when incorporated onto the probe is that it is capable of being ligated to the 5′ end of the probe by DNA ligase. A suitable ligatable terminator is 3′ amino dNTP. The 3′ amino group is a substrate for DNA ligases (U.S. Pat. No. 5,593,826) but acts as a terminator for DNA polymerases. Therefore, it is not possible to add more than one nucleotide to the 3′ end of the probe.

If the correct nucleotide is present in the probe at the site of the SNP, there will be only one base mismatch (at −1 to −4, where the deliberate mismatch has been located) in the system and the DNA polymerase will efficiently incorporate the next base to fill the gap, i.e. the terminator nucleotide. If the incorrect nucleotide is present in the probe at the site of the SNP there will be two base mismatches in the system (at the SNP site and at −1 to −4 as above). The presence of the two mismatches so close to the 3′ end of the probe significantly inhibits DNA polymerase activity and hence the gap is filled with the terminator nucleotide to a much-reduced degree.

The product of the DNA polymerase reaction is then treated with DNA ligase and the effect of any mismatch is greatly enhanced. When there are two mismatches present (i.e. the mismatched SNP base and the deliberate mismatch) the level of gap filled product is reduced and that which is present still has two mismatches close to the 3′ end of the probe. This configuration is not conducive to ligation to the 5′ end of the probe by DNA ligase.

If there is more than one nucleotide gap between the 3′ and 5′ end of the probe after hybridisation to the target, then the gap may be filled without using a terminator nucleotide. There is no essential requirement to use a terminator nucleotide. As the sequence of the bases in the gap region complementary to the target sequence will be known it is possible to use a nucleotide composition that includes dNTPαS or the dNTPs or NTP's at very low concentration. One skilled in the art will also appreciate that any nucleotide that acts as a terminator for DNA polymerases and a substrate for ligases could be utilised within the invention. The DNA polymerase for the gap filing reaction is suitably selected from E Coli DNA polymerase I (Klenow fragment), E Coli DNA polymerase I (Stoffel fragment), T7 DNA polymerase, Pyrococcus furious DNA polymerase, Bacillus stearothermophilus DNA polymerase or the DNA polymerase activity of reverse tnanscriptases such as AMV reverse transcriptase with T7 DNA polymerase being preferred. Ideally the polymerase used will have a low proeessivity such that it will detach from the template rapidly, permitting access for the DNA ligase.

Thus the invention provides a single stranded oligonucleotide probe capable of hybridising to a target nucleic acid sequence comprising

a) a region at the 5′ end of said oligonucleotide which hybridises to the target nucleic acid sequence.

b) a region at the 3′ end of said oligonucleotide which hybridises to an adjacent region of the target nucleic acid sequence which produces a gap of up to 10 nucleotide between the 3′ and 5′ end of said oligonucleotide when hybridised to the target nucleic acid sequence.

c) an allele-specific base at the 3′ end of said oligonucleotide.

d) a deliberate base mismatch to the target nucleic acid sequence located up to 4 nucleotides from the 3′ end of said oligonucleotide.

e) a phosphate group at the 5′ end of the oligonucleotide.

The method of the invention may be carried out with two allele-specific probes, each with a different backbone sequence to facilitate its selective amplification with one of two pairs of generic, probe-specific ERCA primers. The 3′ terminal base is SNP-specific. The 5′ end is phosphorylated. When hybridised to its target the probe ends are separated by a single base gap corresponding to the nucleotide immediately 3′ of the SNP site. A deliberate mismatch is designed into the probe sequence 2 bases 5′ of the allele discriminating [SNP] base.

Preferably, the SNP analysis using ERCA should interrogate both alleles of an SNP of interest in the same reaction, i.e. in a single microtitre plate well, and should score the result using a detectable end point that is coupled to a suitable detection system. Fluorescence is ideally suited as the end point and is the detection mechanism of choice. It is important that the assay can “call” the differences between a homozygote wild type, heterozygote and homozygote mutant. Allele calling must be robust enough to accommodate a range of mismatches in different sequence contexts. It is essential to maximise allele discrimination by reducing signal from the incorrect allele. It should be apparent to one skilled in the art that the method of the invention is equally applicable to interrogating single and multiple (>2) alleles in the same assay. The invention can also utilise methods of detection other than fluorescence such as mass spectrometry, radioactivity and others obvious to one skilled in the ari

For the purposes of the examples cited here it will be assumed that a −3 mismatch is used Probes according to the preferred embodiment are hybridised to a nucleic acid target e.g. genomic DNA, PCR product, RNA or mRNA targets. The ‘correct’ duplex pairing of probe 1 with allele 1 will carry a single dehbberate mismatch at the −3 position. DNA polymerase will efficiently prime synthesis from an oligo bearing a mismatch at the −3 position. The incorrect probe/template combination will have two of its 3 terminal bases mismatched, namely −1 and −3, where −1 represents the SNP mismatch. This arrangement significantly reduces the ability of a DNA polymerase to initiate chain extension.

A DNA Polymerase and a dNTP cocktail are added. The dNTP mix contains a low concentration of the amino-dNTP to complement the base 3′ of the SNP plus normal levels of the other three dNTPs. The polymerase extends the matched probe 3′—OH by adding the amino-dNTP, an event that terminates polymerisation on this template. Multiple base additions that might result in strand displacement of the adjacent 5′ end of the probe are thus prevented. Strand displacement can be a significant problem in some gap fill-in strategies.(e.g. as described in EP 439182).

The mismatched template/probe duplex is extended with a much lower efficiency owing to its double mismatch. Hence, there is a high degree of selection in favour of the correct probe/allele pairing. At the next stage, DNA ligase is employed to further discriminate against that small fraction of mismatched probes where extension has taken place.

Polymerase fidelity is favoured by the use of amino dNTPs. When the enzyme adds a ‘correct’ base, (i.e. amino-dNTP), it creates a nicked duplex for DNA ligase to act upon. If the enzyme adds an incorrect, non-amino dNTP, there is no chain termination and it can continue to insert farther bases by strand displacing the 5′ end of the probe. The resulting flap structure cannot be subsequently ligated and so polymerase mis-incorporation events are selected against

DNA ligase accepts the 3′ terminal NH₂ group of the matched template/probe nicked duplex. Even the ‘matched’ duplex will contain a single mismatch, now at the 4 position, but this should not significantly affect ligation efficiency. However, any mismatched probe that were falsely extended 1 base by polymerase in the preceding stages will now represent very poor substrates for the ligase because they will contain both a −2 and a −4 mismatch DNA or RNA ligase from a variety of sources e.g. Thermus aquaticus, Thermus thermophilus, or bacteriophage T4 may be used in the method, with bacteriophage T4 DNA ligase being preferred

The ligated product may now be detected in a variety of ways but methods based on rolling circle amplification are preferred. This allows rapid, sensitive and homogeneous detection of the ligated product and therefore allows the identity of the base at the site of polymorphism to be determined.

This dual selection by polymerase and ligase minimises erroneous ligation events that are otherwise readily amplified in conventional ERCA This is predicted to significantly improve the overall assay signal to noise ratio and sensitivity.

The ERCA reaction will not be affected by 3′ amino dNTPs as the addition of 3′—OH dNTPs at 400 μM in the amplification mixture effectively dilutes them.

### EXAMPLES

### Example 1

### Use of 3′ Amino TTP as a DNA Polymerase Chain Terminator

Chain terminator sequencing reactions were carried out using either 3′ amino dTTP or ddTTP. Reactions (20 μl) contained 1× Sequenase™ reaction buffer (Amersham Pharmacia Biotech), 40 U Sequenase™ V2.0 DNA polymerase, 0.5 pmol of M13mp18+ strand ssDNA template, 1 pmol 5′ ³² P-labeled universal sequencing primer, 7 μM 3′ amino dTTP, 0.8 μM or 8 W dTTP, 2 μM dCTP, 2 μM dGTP and 2 μM dATP. Control reactions contained 7 μM ddTTP in place of 7 μM 3′ amino dTTP. Reactions were incubated at 37° C. for 10 minutes. 3 μl was heat denatured in formamide loading dye and electrophoresed on an 8% polyacrylamide/urea sequencing gel. The gel was exposed to a phosphor screen, scanned and the image captured using a Molecular Dynamics ‘Storm’ PhosphorImager.

3′ amino TMP gave the same sequencing ladder as ddTTP with correct terminations at the first 20 templated A positions downstream from the universal primer thereby showing that 3′ amino TTP was an effective substrate for DNA polymerase and capable of chain termination.

### Example 2

### Ligation and Circularization of an Amino Terminated Probe by DNA Ligase

The linear probe 5′AAGAAACCATGTAGTTTGTATTCGAATGTCCTATCCTC AGCTGTCAGAACTCACCTGTTAGACGTCGATCTCTCTCTAGTGGAAGTTAGCT- NH₂3′, (Seq Id No 1) bearing a 3′ terminal amino group, was synthesized using standard phosphoramidite chemistry. 25 nmol of probe was phosphorylated at the 5′ end in 1×PNK buffer with 6 μl T4 polynucleotide kinase (Life Technologies Inc.) and 4 μl of 10 mCi/ml, 6000 Ci/mmol ³²PγATP (Amersham Pharmacia Biotech) for 30 minutes at 37° C. in a 100 μl reaction. The linase was inactivated at 80° C. for 5 min. Annealing reactions (50 μl) were setup containing 20 nM ³²P labeled probe, 100 nM complementary oligonucleotide 5′ATACAAACTACATGGTITCTTAGCTA ACTTCCACTAGAGAGAGA 3′ (Seq Id No 2) and 1×T4 DNA ligase buffer (New England Biolabs). These were heated at 95° C. for 2 minutes then cooled slowly to room temperature over 1.5 hours. 1, 2 or 5 Weiss units of T4 DNA ligase (New England Biolabs) were added after annealing. Ligation reactions were incubated at 37° C. overnight Next, linear and un-ligated probe molecules were removed by digestion with 10U Exonuclease I plus 50U T7 Gene 6 Exonuclease (Amersham Pharmacia Biotech) for 1 h at 37° C. for 0, 1 or 3 hours. ³²P labeled products were resolved on an 8% denaturing polyacrylamide gel. After exposure overnight on a Molecular Dynamics ‘Storm’ PhosphorImager ligated products with a mobility identical to that of a circular probe size marker were observed in all reaction containing T4 DNA ligase. This showed that 3′ amino terminated probes were substrates for, and can be ligated by, DNA ligase.

### Example 3

### Ligation and Circularization by DNA Ligase of a Probe Extended and Terminated by DNA Polymerase and 3′ Amino TTP

50 μl annealing reactions were performed as in Example 2 containing 100 nM linear probe 5′AAGAAACCATGTAGTTTGTATTCGAATGTCCTATCCTCAGCTGTCAGAACTC ACCTGTTAGACGTCGATCTCTCTCTAGTGGAAGTTAGC 3′, (Seq Id No 3), 200 nM complementary oligonucleotide target 5′ATACAAACTACATGGTTTCITAGCTAACTTCCACTAGAGAGAGA 3′ (Seq Id No 4) and 1× Sequenase™ buffer. The linear probe and complementary oligonucleotide target were designed such that, when annealed, the probe ends are held together separated by a single base gap opposite an A in the target strand. This gap was filled by adding 32.5 U Sequenase™ V2.0 DNA polymerase containing yeast inorganic pyrophosphatase (Amersham Pharmacia Biotech) and 10 μM 3′ amino TTP and incubating at 37° C. for 10 minutes. The polymerase was heat inactivated at 70° C. and ligation with T4 DNA ligase performed as in Example 2.

Gel analysis revealed that reactions without DNA polymerase gave a ³²P labeled species corresponding to linear probe. Reactions with DNA polymerase yielded a product 1 nucleotide larger due to the 3′ terminal addition of 3′ amino TTP. Reactions with DNA ligase but no DNA polymerase also gave a product larger than the initial probe due to the addition of an adenylate moiety at its 5′ end by ligase. A circularized probe product having much slower gel mobility than the linear species was observed in those reactions that had both ligase and polymerase present.

This demonstrates that DNA polymerase can utilize 3′ amino TTP to fill-in a gapped circular probe and that the resultant nicked circle can be successfully ligated by DNA ligase forming a covalently closed, single stranded, circular DNA molecule which may act as a template for Rolling Circle Amplification.

### Example 4

### Rolling Circle Amplification of Circular DNA Probes Containing 3, Amino TTP

Circularized probes were prepared by ligation according to Examples 2 and 3. ie) ligation of a padlock pre-synthesized with a 3′ amino T residue or addition of 3′ amino TIP to a gapped probe by Sequenase™ followed by ligation.

30 μl ERCA reactions contained 5 μl ligation reaction, 1×Bst DNA polymerase buffer (New England Biolabs), 5% v/v DMSO, 400 μM each of dATP/dTTP/dGTP/dCTP, 1 μM primer 5′CAGCrGAGGATAGGACATTCGA 3′, (Seq Id No 5), 1 μM primer 5′TCAGAACTCACCTGTTAGACG 3′ (Seq Id No 6), and 8 U Bst DNA polymerase. Reactions were incubated at 65° C. for 2 hours and then analysed by agarose gel electrophoresis. Probes circularized by either method produced large amounts of characteristic RCA product ranging in size from 90 base pairs to >23 kb. Thus, the presence of a 3′ amino nucleotide in the circularized probe does not adversely affect Rolling Circle Amplification.

1. A single strand oligonucleotide probe capable of hybridising to a target nucleic acid sequence comprising
a) a region at the 5′ end of said oligonucleotide which hybridises to the target nucleic acid sequence.
b) a region at the 3′ end of said oligonucleotide which hybridises to an adjacent region of the target nucleic acid sequence which produces a gap of up to 10 nucleotides between the 3′ and 5′ end of said oligonucleotide when hybridised to the target nucleic acid sequence.
c) an allele-specific base at the 3′ end of said oligonucleotide.
d) a deliberate base mismatch to the target nucleic acid sequence located up to 4 nucleotides from the 3′ end of said oligonucleotide.
e) a phosphate group at the 5′ end of the oligonucleotide.

2. A method of determining the identity of a base at a specific site in a target nucleic acid sequence comprising the steps of
a) incubating the target nucleic acid with a probe of claim 1 to produce a gap of up to 10 nucleotides between the 3′ and 5′ ends of the oligonucleotide.
b) incubating the product of step a) with a DNA polymerase and a nucleotide composition capable of filling the gap of step a).
c) ligating the product of step b) with a DNA ligase.
d) detecting the ligation product of step c).

3. A method according to claim 2 wherein the DNA polymerase and nucleotide composition capable of filling the gap of step a) does not allow synthesis beyond the gap.

4. A method of claim 2 or 3 that further includes the detection of the ligation product of step c) by means of roiling circle amplification.

5. A method of claims 2 to 4 wherein the gap between the 3′ and 5′ ends of the oligonucleotide when hybridized to the target nucleic acids is 1 base.

6. A method according to claims 2 to 4 wherein nucleotide composition of step b) comprises a terminator nucleotide capable of being ligated by DNA ligase.

7. A method of claim 6 wherein the terminator nucleotide is a 3′NH₂-dNTP.

8. A method of claim 2 where the ligase is either a DNA ligase or an RNA ligase.

9. A method of claim 2 where ligation is non-enzymatic or chemically induced.
